(19) United States
(12) Patent Application Publication
(10) Pub. No.: US 2016/0184539 A1
(43) Pub. Date: Jun. 30, 2016

(54) CPAP DEVICE AND BLOWER UNIT FOR CPAP DEVICE

(71) Applicant: NIDEC COPAL ELECTRONICS CORPORATION, Tokyo (JP)

(72) Inventors: Takashi SUZUKI, Iruma-shi (JP); Yasuhiro TOBINAI, Iruma-shi (JP); Takatoshi INOGUCHI, Iruma-shi (JP); Takayuki ENDO, Iruma-shi (JP); Masatoshi OBAYASHI, Iruma-shi (JP); Naoya EGUCHI, Iruma-shi (JP)

(73) Assignee: NIDEC COPAL ELECTRONICS CORPORATION, Tokyo (JP)

(21) Appl. No.: 14/910,274

(22) PCT Filed: Jul. 29, 2014
(86) PCT No.: PCT/JP2014/003970

§ 371 Date: Feb. 5, 2016

(30) Foreign Application Priority Data
Aug. 9, 2013 (JP) 2013-166391

(51) Int. Cl. A61M 16/00; A61M 16/06

(52) U.S. Cl. CPC A61M 16/0066; A61M 16/06; A61M 2205/42

(57) ABSTRACT

The present invention relates to a CPAP device and a blower unit, and achieves the compatibility between reductions in size and in weight and noise reduction in a high order. A blower unit 10 includes a casing 11 including an air suction port 111, a fan 50 which has an air receiving port 531 and an air sending port 542, is provided with a fluid dynamic bearing, causes air to be suctioned from the air suction port 111 to receive the air from the air receiving port 531 and sends out the air from the air sending port 542, and an emission silencer 17 which is coupled to the air sending port 542 and reduces sounds as the air flows which air is sent out from the air sending port 542 by the fan 50.

### TECHNICAL FIELD

The present invention is related to a CPAP (Continuous Positive Airway Pressure) device which is used for treatment of Sleep Apnea Syndrome and a blower unit which is included in the CPAP device.

### BACKGROUND ART

For treatment of Sleep Apnea Syndrome, there have been used CPAP devices which forcibly send air into the respiratory tract by a fan while putting a nasal cannula or a mask on a face. As such a CPAP device, there has been generally adopted a configuration in which a main unit which includes a fan, a control section and the like is placed at a position away from a human body, and between the main unit and the mask or the like which is put on a face is connected by a hose of about 1.5 meters and air is sent in through the hose. Nasal cannulas or masks which have various shapes and are formed by various materials have been developed and put onto the market, and a patient arbitrarily chooses and uses a mask which fits for its face shape and matches its preferences.

In a case of a CPAP device of such configuration, since there are a number of problems such as ones in which the device requires a hose having a length as long as 1.5 meters, its main unit has a volume of the order of 140 mm×180 mm×100 mm and it has a size inconvenient for carrying, and such device is inconvenient for a patient to handle it, contrary to that the treatment method is required to be used every day, such device becomes one of treatment devices which are often not used.

For this reason, it has become a problem how to reduce the size and the weight of such CPAP device.

In addition, in a CPAP device, a fan is rotated according to breathing of a patient, air flows as the fan rotates, and sounds are produced as the fan rotates and the air flows. Since a CPAP device is a device which is used while a patient is sleeping, it is especially required to be silent, and how to reduce the sounds becomes a problem.

As reductions in size and in weight of the CPAP device are advanced, it is expected that the CPAP device is configured such that its main unit is placed immediately close to a patient and is connected to its mask with a hose having a short length as compared to conventional cases, like that the main unit is placed at, for example, near a pillow of a patient while sleeping, or it is put in a breast pocket of a patient, and so on. In such cases, since noise sources come near a human head, noise reduction also becomes a further big problem.

As a proposal in which noise reduction is aimed for a CPAP device, for example, the Patent Literature 1 discloses providing a chamber to reduce noise.

However, in this case, the chamber itself becomes large-sized, and thus, the problem as to reduction in size of the CPAP device may not be eliminated.

In addition, the Patent Literature 2 discloses a configuration in which an inlet silencer and an outlet silencer are arranged at an inlet side and an outlet side in a blower, respectively.

However, the Patent Literature 2 does not describes any concrete configuration and material of the inlet silencer and the outlet silencer, and in addition, it does not appear to be a proposal in which reduction in size as a whole including the blower is considered.

Incidentally, in the present invention, which will be described later, an air dynamic pressure bearing, which is a form of the fluid dynamic bearing, is used, and there are listed in here literatures (Patent Literatures 3 and 4) in which fans including air dynamic pressure bearings are disclosed.

### PRIOR ART LITERATURES

### Patent Literatures

Patent Literature 1: Japanese Laid-open Patent Publication No. H7-275362
Patent Literature 2: Japanese National Publication of International Patent Application No. 2002-537006
Patent Literature 3: Japanese Laid-open Patent Publication No. 2007-57048
Patent Literature 4: Japanese Laid-open Patent Publication No. 2009-52485

### ABSTRACT OF THE INVENTION

### Technical Problem

In view of the foregoing, it is an object of the present invention to provide a CPAP device in which the compatibility between reductions in size and in weight and noise reduction is achieved in a high order and a blower unit for the CPAP device.

### Solution to Problem

A CPAP device to obtain the above-described object includes:

a blower unit that includes:

a casing which has an air suction port;

a fan which has an air receiving port and an air sending port, is provided with a fluid dynamic bearing, causes air to be suctioned from the air suction port to receive the air from the air receiving port and sends out the air from the air sending port; and

an emission silencer which is coupled to the air sending port and reduces sounds as the air flows which air is sent out from the air sending port by the fan, wherein

an air intake port of a nasal cannula or a mask which has the air intake port, is attached to the head of a patient to cover an external naris or a nose of the patient and supplies air taken in from the air intake port to a respiratory tract of the patient and the blower unit are coupled with a hose, and the air sent out from the blower unit is sent to the nasal cannula or the mask.

The fan including the fluid dynamic bearing is used in the CPAP device according to the present invention. The fan may be rotated significantly faster compared to a fan which is conventionally applied to a CPAP device. For this reason, a diameter of a blade required to obtain a required pressure and a required air flow volume is greatly reduced, and in addition, the weight is also significantly reduced. In a CPAP device of conventional type, as one example, a fan which includes a blade having a diameter of 53 mm and has a weight of approximately 240 g is used, and if a fan of fluid dynamic bearing is applied, for example, a fan which has a blade having a diameter of 29 mm and has weight of approximately 40 g may only be required.

However, in a case in which a fan of the fluid dynamic bearing is applied, the fan is required to be rotated faster compared to a conventional fan, specifically at the time of inspiration, it is required to further increase the rotation speed in order to increase the air flow volume, and thus noise becomes large. It is observed that the noise is transmitted from a sending side of the fan through a flow path to a patient.

In addition, since an amount of changing of the rotation speed of the fan also increases as the air flow amount changes by breathing of a patient, changing of the noise by rotation changing amount increasing of the fan (changing of frequencies of the noise and changing of noise levels) also increases, and thus resulting in more harsh noise.

Accordingly, in the present invention, reduction in size and reduction in weight are obtained by applying a fan of the fluid dynamic bearing, and simultaneously, an emission silencer is provided at a side of sending out air, and with this, a CPAP device in which the compatibility between reductions in size and in weight and noise reduction is achieved in a high order is obtained.

Here, in the CPAP device according to the present invention, it is preferable that the emission silencer is a silencer which includes a sound absorbing material made of foaming material.

The emission silencer is also reduced in size and in weight by forming the emission silencer with the sound absorbing material made of foaming material, and thus the CPAP device as a whole is further reduced in size and in weight.

Further, in a case in which the sound absorbing material made of foaming material is used, since there are effects of reducing noise of a broad frequency band, compared to the chamber configuration described in the Patent Literature 1, it is specifically effective to noise including broad frequency components such as wind noise.

In addition, in the CPAP device according to the present invention, it is preferable that the blower unit further includes a suction silencer that includes a sound absorbing material in which a suction flow path to guide the air suctioned from the air suction port to the air receiving port is formed, and that supports the fan such that the suction silencer enfolds the fan with the sound absorbing material.

When the sound absorbing material is included and the suction silencer to support the fan such that the suction silencer enfolds the fan is provided, a CPAP device in which both of noise as air is suctioned and vibrations of the fan are reduced is obtained.

Further, in the CPAP device according to the present invention, it is preferable that the air sending port and the emission silencer are connected with a joint formed with an elastic body.

When between the air sending port of the fan and the emission silencer is connected by the joint formed with the elastic body, vibration transmission of the fan to the emission silencer is reduced, and noise is further reduced.

In addition, a blower unit to obtain the above-describe object includes:

a casing which has an air suction port;

a fan which has an air receiving port and an air sending port, is provided with a fluid dynamic bearing, causes air to be suctioned from the air suction port to receive the air from the air receiving port and sends out the air from the air sending port; and

an emission silencer which is coupled to the air sending port and reduces sounds as the air flows which air is sent out from the air sending port by the fan, wherein

the blower unit sends air into a hose which is coupled to an air intake port of a nasal cannula or a mask which has the air intake port, is attached to the head of a patient to cover an external naris or a nose of the patient and supplies the air taken in from the air intake port to a respiratory tract of the patient.

Incidentally, the hose in the present invention is not limited to a hose which simply has a function as a flow path, and includes also what has an other function in addition to that of a flow path, for example, such as connecting a fan with a mask via a humidifying unit, and is substantially considered as a hose.

### Advantageous Effects of Invention

According to the CPAP device and the blower unit of the present invention, the compatibility between reductions in size and in weight and noise reduction is achieved in a high order.

### BRIEF DESCRIPTION OF THE DRAWINGS

FIG. 1 is an external appearance view of a whole configuration of a CPAP device as a first embodiment.

FIG. 2 is an explanatory view illustrating an example of usage conditions of the CPAP device illustrated in FIG. 1.

FIG. 3 is an exploded perspective view of the CPAP device according to the first embodiment whose external appearance is illustrated in FIG. 1.

FIG. 4 is a transparent view of the CPAP device according to the first embodiment when viewed from obliquely above.

FIG. 5 is a sectional view along arrows A-A illustrated in FIG. 4 of the CPAP device according to the first embodiment.

FIG. 6 is a transparent view when the casing and the suction silencer are removed from the CPAP device according to the first embodiment, and a fan, an emission structure body and the like are viewed from obliquely above.

FIG. 7 is a control block diagram of the CPAP device according to the present embodiment.

FIG. 8 is an external appearance perspective view of a turbofan used in the CPAP device according to the first embodiment.

FIG. 9 is a plan view of the turbofan.

FIG. 10 is an exploded perspective view of the turbofan viewed from obliquely above.

FIG. 11 is an exploded perspective view of the turbofan viewed from obliquely below.

FIG. 12 is a view illustrating a blade 529 which is a part of the turbofan.

FIG. 13 is a sectional view of the turbofan in a direction indicated by arrows A-A in FIG. 9.

FIG. 14 is a schematic diagram of an experimental equipment.

FIG. 15 is a view illustrating noise of a fan of comparative example and noise of a fan of embodiment example when the pressure is 1.2 kPa and the flow amount is 50 L/min (litter/minute).

FIG. 16 is a view illustrating noise of the fan of comparative example and noise of the fan of embodiment example when the pressure is 1.2 kPa and the flow amount is 110 L/min.

FIG. 17 is a view illustrating noise of the fan of comparative example at the time when breathing stops and at the time of inspiration.

FIG. 18 is a view illustrating noise of the fan of embodiment example at the time when breathing stops and at the time of inspiration.

FIG. 19 is a view illustrating differences between noise levels of the fan of embodiment example and noise levels of the fan of comparative example at the time when breathing stops.

FIG. 20 is a view illustrating differences between noise levels of the fan of embodiment example and noise levels of the fan of comparative example at the time of inspiration.

FIG. 21 is a view illustrating changing of noise levels at the time of inspiration when a length of a sound absorbing material of an emission silencer is changed.

FIG. 22 is a view illustrating noise levels at 7 kHz with respect to the length of the sound absorbing material included in the emission silencer which noise levels are read and obtained from FIG. 21.

FIG. 23 is a view illustrating changing of noise levels at the time of inspiration when the thickness of the sound absorbing material of the emission silencer is changed.

FIG. 24 is a view illustrating noise levels at 1 kHz which noise levels are read from FIG. 23.

FIG. 25 is a view illustrating noise levels at 3.5 kHz which noise levels are read from FIG. 23.

FIG. 26 is a view illustrating noise levels at 5.5 kHz which noise levels are read from FIG. 23.

FIG. 27 is a transparent view when a casing and a suction silencer are removed from a CPAP device according to a second embodiment, and a fan, an emission silencer and the like are viewed from obliquely above.

FIG. 28 is an exploded perspective view of a CPAP device according to a third embodiment.

FIG. 29 is a sectional view of a blower unit of the CPAP device whose exploded perspective view is illustrated in FIG. 28.

FIG. 30 is a sectional view of a fan and an emission silencer of a CPAP device according to a fourth embodiment.

FIG. 31 is a sectional view of a fan and an emission silencer of a CPAP device according to a fifth embodiment.

### BEST MODE FOR CARRYING OUT THE INVENTION

In the following, embodiments of the present invention will be described.

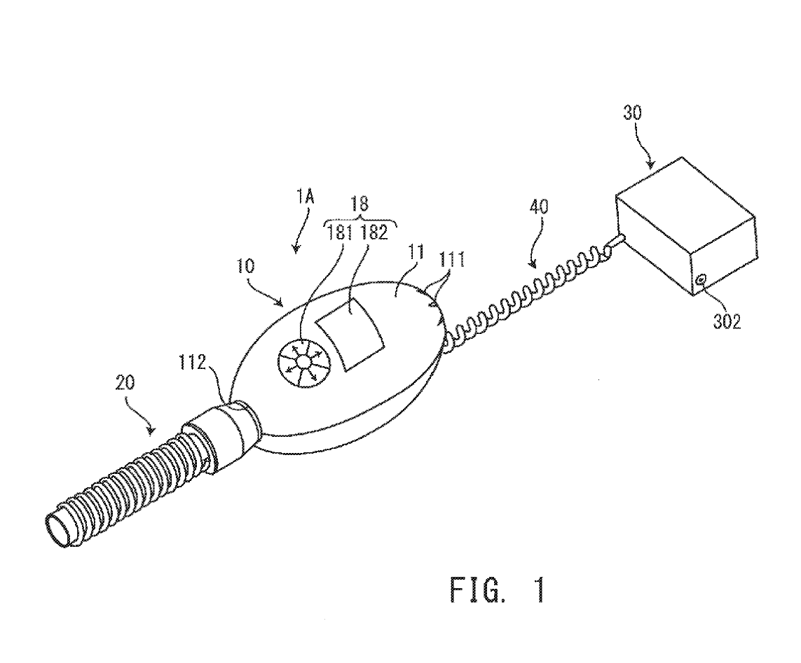

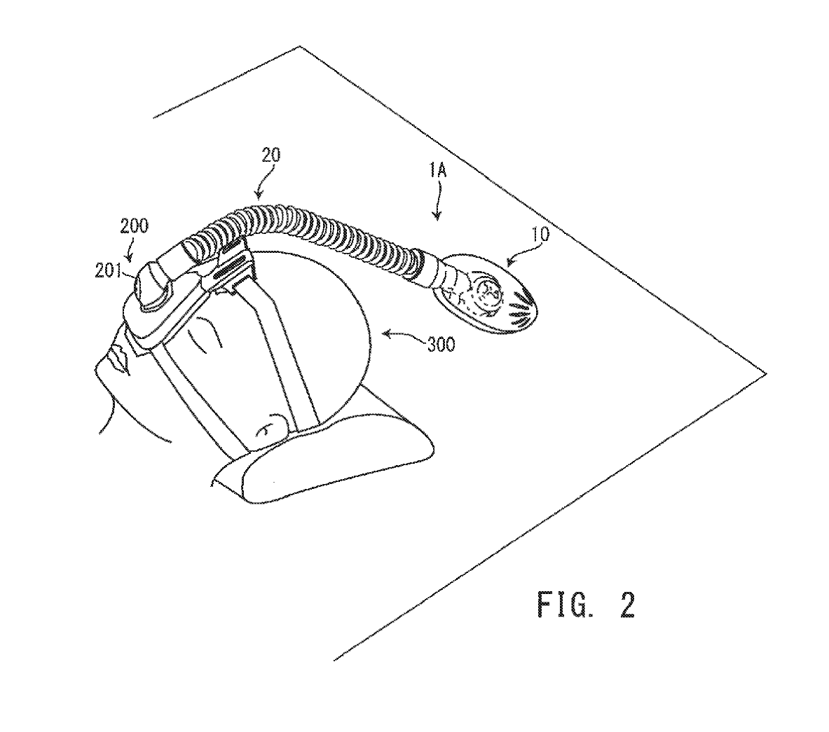

FIG. 1 is an external appearance view of a whole configuration of a CPAP device as a first embodiment according to the present invention, and FIG. 2 is an explanatory view illustrating an example of usage conditions of the CPAP device illustrated in FIG. 1. However, in FIG. 2, illustrations of a battery case 30 and a cable 40 which are illustrated in FIG. 1 are omitted. In addition, in this FIG. 2, with respect to a blower unit 10, a transparent view indicating an outline of an inside thereof is illustrated.

This CPAP device 1A includes the blower unit 10, a hose 20, the battery case 30 and the cable 40. The CPAP device 1A is used, as illustrated in FIG. 2, in a condition in which the blower unit 10 and a mask 200 are connected by the hose 20, the mask 200 is attached on a face of a patient 300 and the blower unit 10 is placed near a pillow. Accordingly, the hose 20 is a hose having a length of, for example, the order of 50 cm. Plural air suction ports 111 are provided in a casing 11 as a casing in which the blower unit 10 is housed, and in addition, a fan which will be described later is provided in the casing 11. When the fan rotates, air is sent into the mask 200 via the hose 20. The air sent into the mask 200 is supplied to a respiratory tract of the patient 300. Breath is emitted to the outside from a leak opening 201 provided in the mask 200. The blower unit 10 according to the present embodiment has an oval spherical shape as a whole, and when the patient 300 wearing the mask 200 changes its posture while keeping its lying posture, for example, when the patient turns over on its bed, a force when the posture is changed is transmitted to the blower unit 10 via the hose 20, the blower unit 10 rolls or slides and thus a position or a posture of the blower unit 10 is also changed according to the posture of the patient.

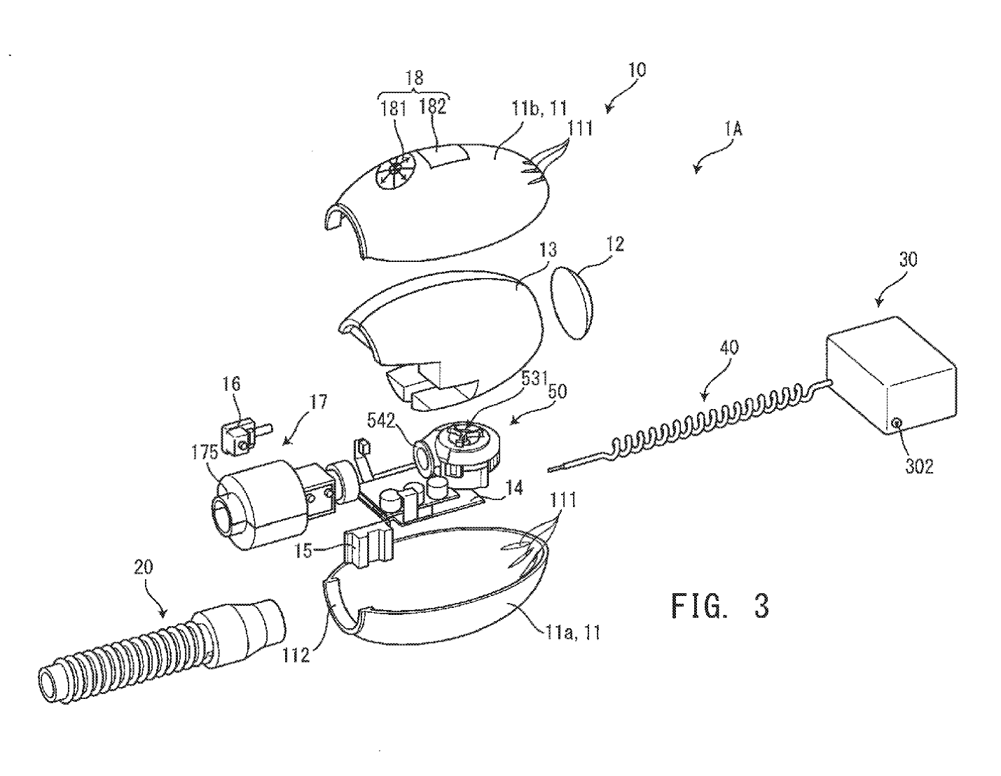

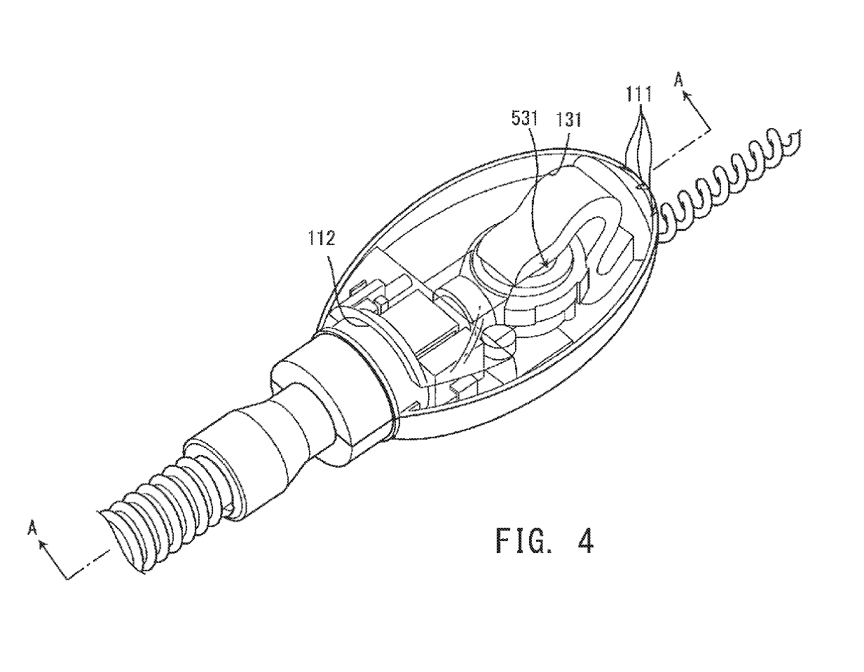

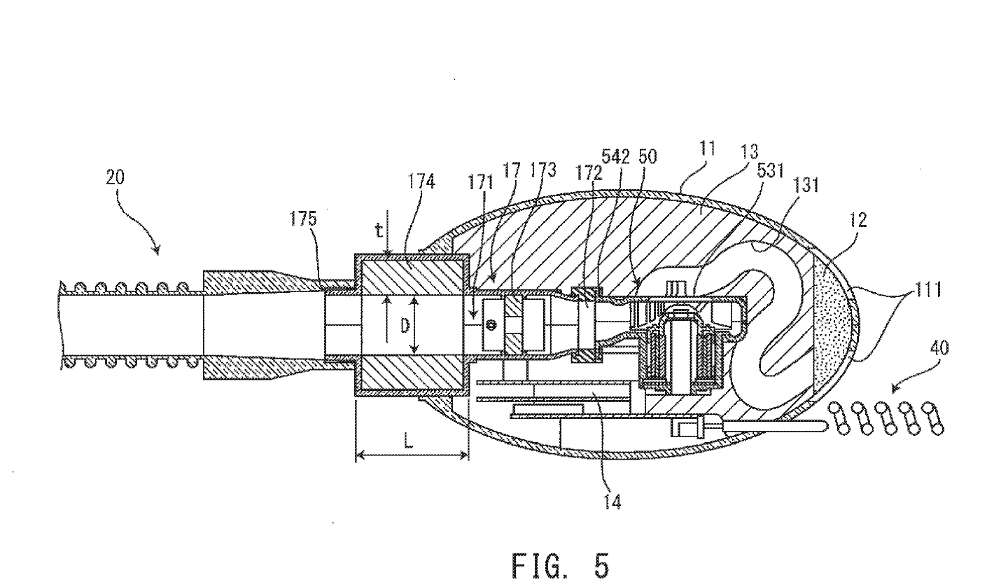

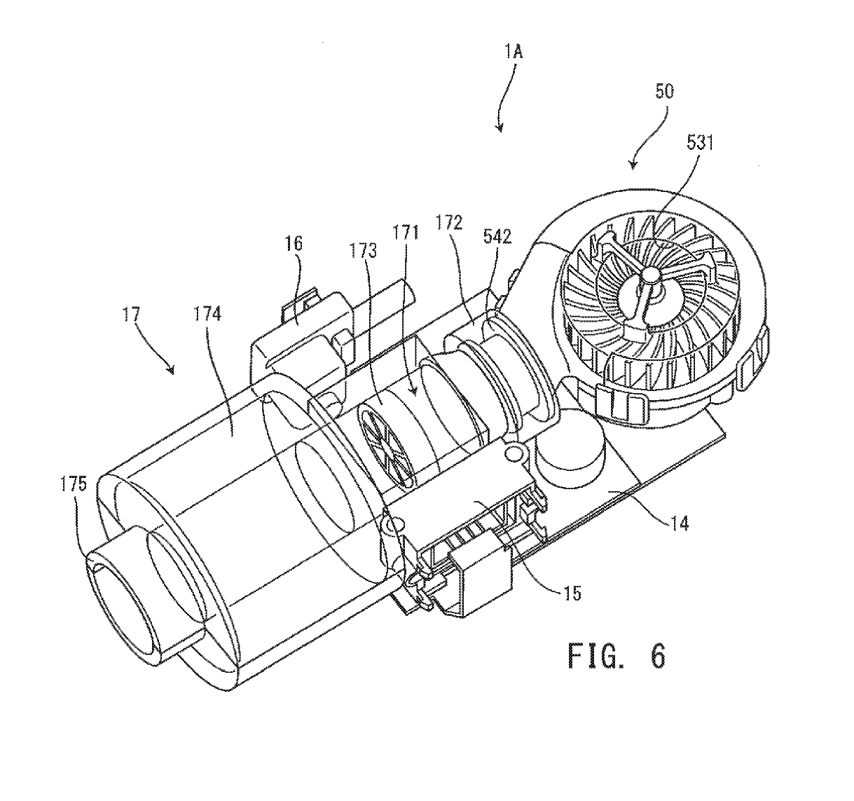

FIG. 3 is an exploded perspective view of the CPAP device according to the first embodiment whose external appearance is illustrated in FIG. 1. In addition, FIG. 4 is a transparent view of the CPAP device according to the first embodiment when viewed from obliquely above, and FIG. 5 is a sectional view along arrows A-A illustrated in FIG. 4 of the CPAP device according to the first embodiment. Further, FIG. 6 is a transparent view when the casing and the suction silencer are removed from the CPAP device according to the first embodiment, and the fan, an emission silencer and the like are viewed from obliquely above.

In the CPAP device 1A according to the first embodiment, the casing 11 of the blower unit 10 is formed by a casing lower section 11a and a casing upper section 11b which are illustrated in FIG. 3.

Since the casing 11 has the oval spherical shape as a whole, the casing 11 easily rolls. In addition, the casing 11 is made of plastic and its external surface is formed to be smooth, and the casing 11 easily moves slidably. In order that the air suction is not disturbed even if the casing 11 rolls or slides, the casing 11 is provided with the plural air suction ports 111.

In addition, the casing upper section 11a is provided with a user interface 18 including an operation button 181 and a display screen 182.

An air filter 12, a suction silencer 13, a control board 14, a flow sensor 15, a pressure sensor 16, an emission silencer 17 and a turbofan 50 as the fan are arranged in the casing 11.

In addition, the CPAP device 1A includes, as described above, the hose 20, the battery case 30 and the cable 40.

The air filter 12 is arranged immediately inside the air suction ports 111 provided in the casing 11, and is a filter which absorbs dusts in the air suctioned from the air suction ports 111.

In addition, the suction silencer 13 has a suction flow path 131 which turns as illustrated in FIG. 4 and FIG. 5, and guides the air suctioned from the air suction ports 111 to an air receiving port 531 of the turbofan 50. The suction silencer 13 plays a role of reducing suction sounds of the air which is suctioned from the air suction ports 111 and guiding the air to the turbofan 50. In addition, the suction silencer 13 supports the turbofan 50 such that the suction silencer 13 enfolds the turbofan 50 with its sound absorbing material, and also plays a role of preventing vibrations of the turbofan 50 from transmitting to the casing 11 and the other remaining members.

The turbofan 50 causes air to be suctioned from the air suction ports 111 of the casing 11, receives from the air receiving port 531 the air which comes through the air filter 12 and the suction silencer 13, and sends out the air from an air sending port 542.

The control board 14 calculates a rotation setting speed of the turbofan 50 according to an initial setting by a doctor or a patient, a flow amount measured by the flow sensor and a pressure measured by the pressure sensor 16, and gives an instruction to the turbofan 50 to rotate at the rotation speed.

The flow sensor 15 and the pressure sensor 16 are sensors which measure a flow amount and a pressure of the air sent out from the turbofan 50, respectively.

Further, the emission silencer 17 is coupled to the air sending port 542 of the turbofan 50 to form an emission flow path 171, and allows the air sent out from the air sending port 542 by the turbofan 50 to be emitted from the blower unit 1A. Between the emission silencer 17 and the air sending port 542 of the turbofan 50 is connected with a joint 172 made of rubber. The joint 172 plays a role of preventing that vibrations of the turbofan 50 are transmitted to the emission silencer 17 to increase noise.

In the emission silencer 17, there are provided a rectifying element 173 and a sound absorbing material 174. The rectifying element 173 is a member which plays a role of regulating a flow of air sent in from the turbofan 50. The flow sensor 15 and the pressure sensor 16 are connected to a downstream side with respect to the flow of air of the rectifying element 173. With this, it is prevented that an unnecessary change by air turbulence is transmitted to the flow sensor 15 or the pressure sensor 16 so that measured values of the air flow or the air pressure are unnecessarily changed.

In addition, the sound absorbing material 174 plays a role of reducing sounds as the air flows which air is sent out from the air sending port 542 by the turbofan 50. The sound absorbing material 174 is a sound absorbing material made of foaming material, for example, urethane foam or EVA (Ethylene Vinyl Acetate) foam. The density of the foaming material is preferably to be within a range of 10 to 100 kg/m³.

The sound absorbing material 174 provided in the suction silencer 17 effectively decreases noise as a patient inspires, as indicated in experimental data which will be explained later. The hose 20 is coupled to an air emission port 175 of the emission silencer 17, and air is sent into the mask 200 via the hose 20.

A battery is housed inside the battery case 30, and electrical power from the battery 301 is supplied to the blower unit 10 via the cable 40. The battery case 30 is provided with a connection terminal 302 to which an AC adapter (not illustrated) for charging the inside battery is connected. A battery is a component having a significant volume and a significant weight, and in order to make the blower unit 10 compact and lightweight, a configuration in which the battery case 30 which is separate from the blower unit 10 is provided and is connected by the cable 40 is applied in here. However, a configuration in which the battery case 30 and the large battery 301 are not provided and an AC adapter is connected to the blower unit 10 to cause the blower unit 10 to operate may be applied.

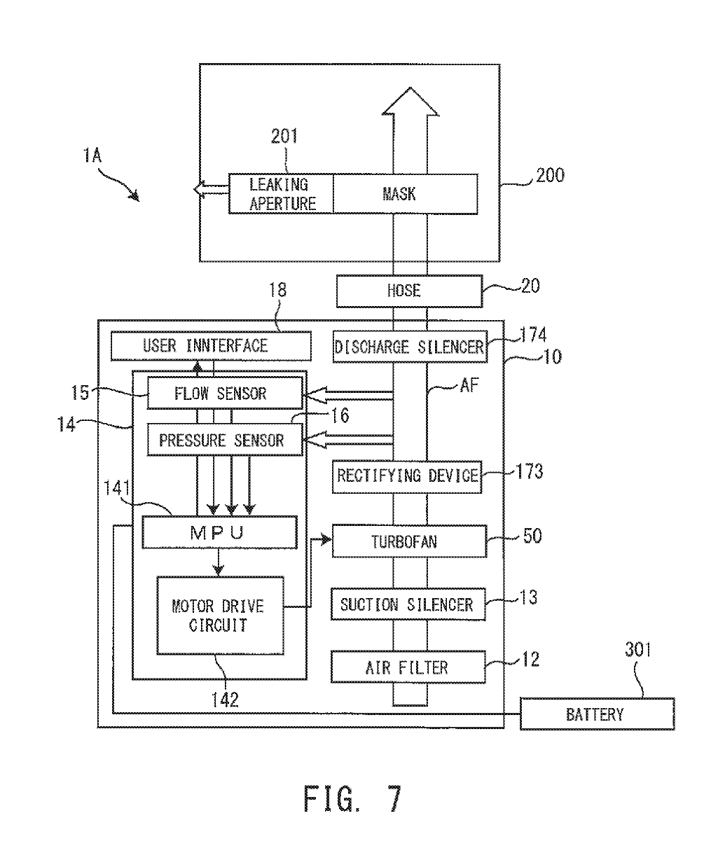

FIG. 7 is a control block diagram of the CPAP device 1A according to the present embodiment.

In here, an air flow path AF which flows from the blower unit 10 via the hose 20 to the mask 200 and a control system of the blower unit 10 are illustrated.

As described above, in the blower unit 10, the air filter 12, the suction silencer 13, the turbofan 50, the rectifying element 173 and the emission silencer 174 are arranged on the air flow path AF, and when the turbofan 50 rotates, air is suctioned from the air suction ports 111 (see, for example, FIG. 4), dusts in the air are removed by the air filter 12, noise as the air is suctioned is reduced by the suction silencer 13, and through the turbofan 50, the air is further regulated by the rectifying element 173, furthermore noise is reduced by the suction silencer 174 and the air is sent into the mask 200 via the hose 20.

The air sent into the mask 200 is sent into a respiratory tract of a patient by inspiration actions of the patient, and is discharged through the leak opening 201 to the outside by expiration actions of the patient.

The blower unit 10 is provided with the user interface 18 including the operation button 181 and the display screen 182 (see, for example, FIG. 1). The patient operates the operation button 181 while checking the display screen 182, and sets a selection between a fixed mode and an automatic mode, a pressure range of air sent out from the turbofan 50 which pressure range is designated from a doctor, on-off timing of the turbofan 50 and the like. Here, the fixed mode is a mode in which a pressure of air sent out from the turbofan 50 is fixed to a designated pressure, and the automatic mode is a mode in which a breathing state of a patient is detected from changes of flow amounts or pressures by the flow sensor 15 or the pressure sensor 16, the pressure is changed in the designated range according to the breathing state of the patient.

Information set by the user interface 18 is input into an MPU (Micro Processing Unit) 141. In addition, air flow amounts and air pressures measured by the flow sensor 15 and the pressure sensor 16 are also input into the MPU 141. The MPU 141 calculates a rotation speed of the turbofan 50 based on those pieces of information. A result of the calculation by the MPU 141 is sent to the motor drive circuit 142, and the motor drive circuit 142 drives the turbofan 50 based on the result of the calculation.

The flow sensor 15, the pressure sensor 16 and the MPU 141 are mounted on the control board 14 (see, for example, FIG. 3) housed in the blower unit 10. Electrical power is supplied to the control board 14 from the battery 301, and electrical power is distributed to each of sections which require the electrical power. In addition, the motor drive circuit 142 is also mounted on the circuit board 14.

One of characteristics of the CPAP device 1A according to the present embodiment is in that the turbofan 50 provided with the air dynamic pressure bearing as a form of the fluid dynamic bearing is applied. Thanks to this, in the CPAP device 1A according to the present embodiment it is succeeded to significantly reduce the size and the weight of the blower unit 10.

Here, the turbofan provided with the air dynamic pressure bearing which is applied to the CPAP device 1A according to the present embodiment will be explained. The turbofan which is explained here is same in terms of the operation principals as those disclosed in the above-described Patent Literatures 3 and 4.

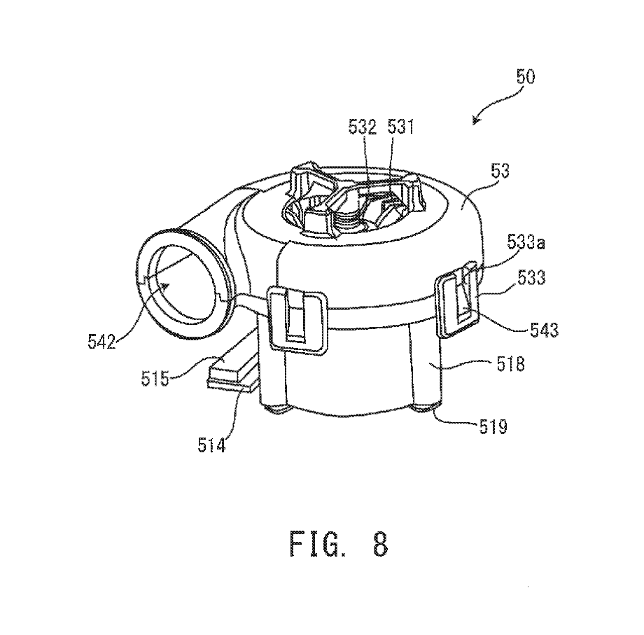

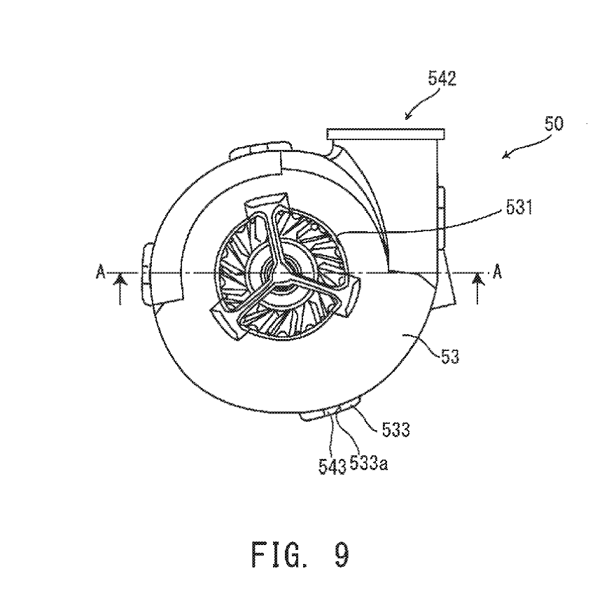

FIG. 8 is an external appearance perspective view of the turbofan used in the CPAP device according to the first embodiment, and FIG. 9 is a plan view of the turbofan.

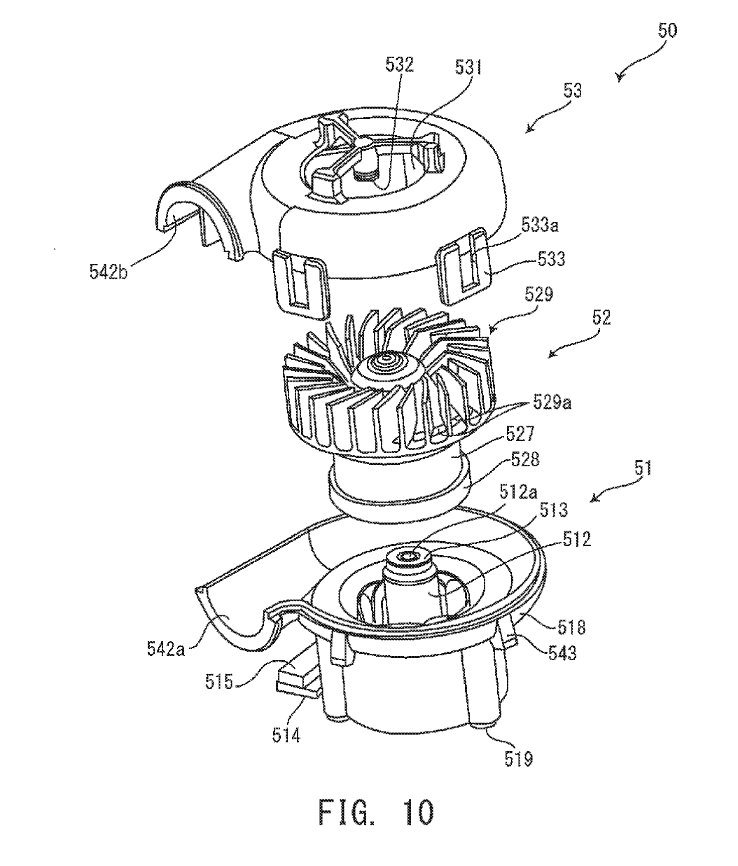

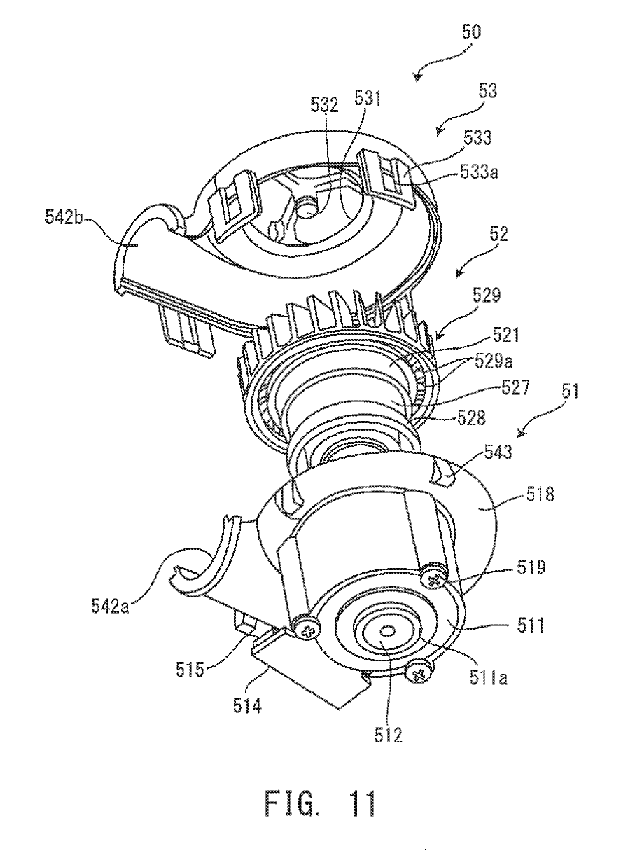

In addition, FIG. 10 and FIG. 11 are exploded perspective views of the turbofan viewed from obliquely above and from obliquely below, respectively.

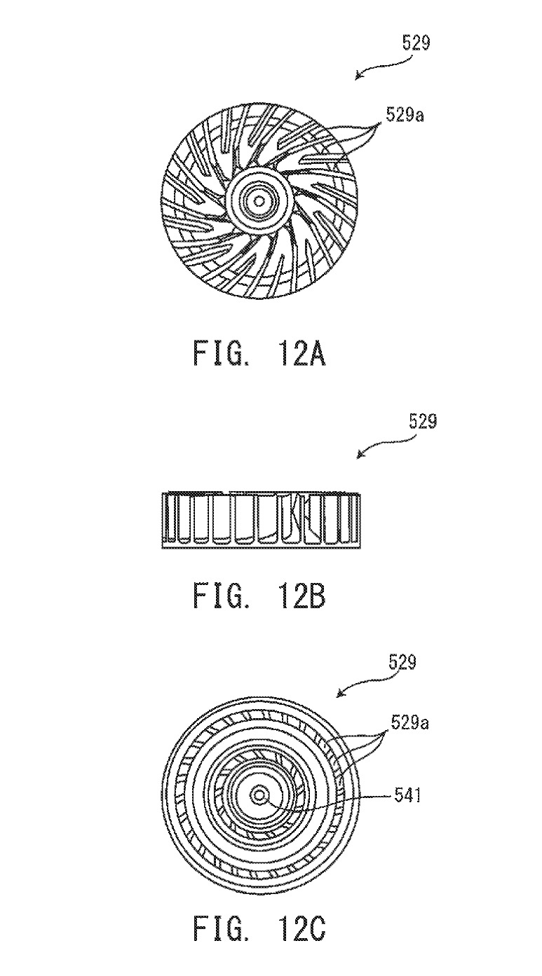

Further, FIG. 12 is a view illustrating a blade 529 which is a part of the turbofan 50. Part (A), part (B) and part (C) of FIG. 12 are a plan view, a side view and a bottom view, respectively.

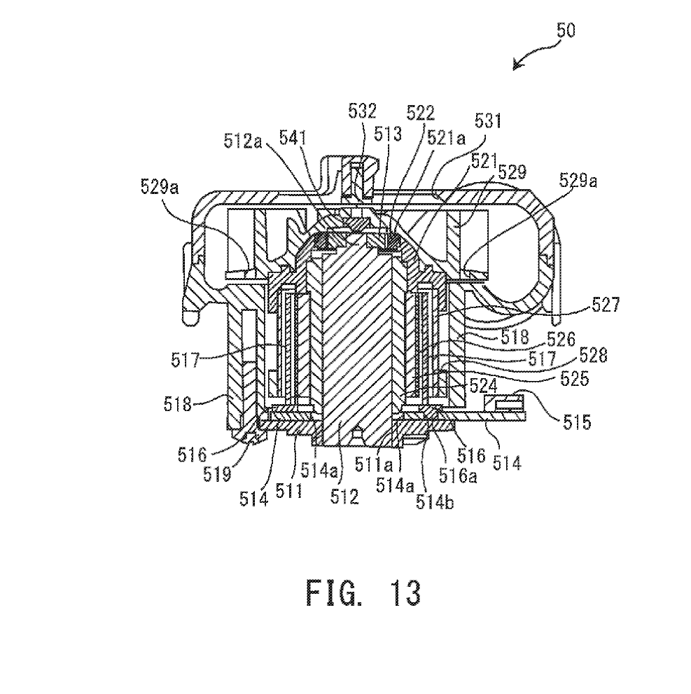

Furthermore, FIG. 13 is a sectional view of the turbofan 50 in a direction indicated by arrows A-A in FIG. 9.

In here, a configuration of the turbofan 50 will be explained while mainly referring to the sectional view of FIG. 13, and referring to other drawings as required.

As illustrated in FIG. 10 and FIG. 11, the turbofan 50 includes, when roughly divided, a stator 51, a rotor 52 and an upper cover 53.

The stator 51 includes a shaft base 511 having ring shape as a base, and is fixed such that a lower section of a shaft 512 fits into an opening 511a in a center of the shaft base 511 having the ring shape. An upper end section 512a of the shaft 512 is formed to have a small diameter, and a thrust magnet (inside) 513 having a ring shape is fixed such that the upper end section 512a fits thereto. In addition, the circuit board 514 is placed on the shaft base 511. The circuit board 514 is formed with an opening 514a to allow the shaft 512 to go therethrough, and spreads to surround the shaft 512. In addition, the circuit board 514 spreads such that a portion thereof is extended off to the outside, and a connector 515 for connecting to an external circuit is arranged on the extended-off portion.

In addition, a coil base 516 having a ring shape which coil base surrounds the shaft 512 while being slightly away from the shaft 512 is placed on the circuit board 514. In the coil base 516, leg sections 516a which go into openings 514b provided in the circuit board 514 and are supported by the shaft base 511 are arranged at plural positions in a circumferential direction. In other words, the coil base 516 has a shape as a whole in which the coil base 516 is supported at the leg sections 516a by the shaft base 511, and circles on an upper surface of the circuit board 514 around the shaft 512 as a center.

Further, a coil 517 which is formed to have a cylindrical shape as a whole is put on the coil base 516, and a lower end of the coil 517 is fixed to the coil base 516. Electrical power of three-phase pulse is supplied to the coil 517.

In addition, a casing 518 is screwed to the shaft base 511 by screws 519.

The rotor 52 has a hub 521 as a base. An opening 521a is formed in an upper section of the hub 521, a thrust magnet (outside) 522 having a ring shape is fixed to an edge of the opening 521a. An internal circumferential surface of the thrust magnet (outside) 522 faces an external circumferential surface of the thrust magnet (inside) 513 across a significantly small gap therebetween, and a contact in a thrust direction between a sintered body 541 and a shaft upper end section 512a is avoided by an absorbing force between their magnetic forces.

In addition, a sleeve 524 having a cylindrical shape is fixed to the hub 521. An internal circumferential surface of the sleeve 524 faces an external circumferential surface of the shaft 512, a significantly small gap in order of μm is formed between the sleeve 524 and the shaft 512.

A magnet 525 is fixed to an external circumferential surface of the sleeve 524, and a reinforcing ring 526 is attached to an external circumferential surface of the magnet 525. Since the rotor 52 of the turbofan 50 rotates in a high speed, there is a possibility in which the magnet 525 is cracked by a centrifugal force, and the reinforcing ring 526 is for preventing such crack. An external circumferential surface of the reinforcing ring 526 faces an internal circumferential surface of the coil 517 across a narrow space therebetween. Further, on a side of an external circumferential surface of the coil 517, a back yoke 527 is arranged with a space between the coil 517 and the back yoke 527. The back yoke 527 forms a magnetic circuit together with the magnet 525 and plays a role of increasing an interaction with the coil 517. A balance ring 528 is fixed to a lower section of the back yoke 527. The balance ring 528 is a member for adjusting a balance when the rotor 52 rotates.

In addition, a blade 529 (see also FIG. 11 together) is fixed to an upper section of the hub 521. The blade 529 is a component which sends out air by the rotation of the rotor 52.

Further, the sintered body 541 is fixed to a lower central section of the blade 529. The sintered body 541 is for causing an air gap between the stator 51 and the rotor 52 to have a damper effect, and since when the rotor 52 is going to move in the thrust direction it is possible to prevent an abrupt movement of the rotor 52 by the damper effect, it makes it possible that the rotor 52 may rotate in a high speed in a non-contact manner with respect to the stator 51. In addition, the sintered body 541 is placed at a position facing the upper end section 512a of the shaft 512 of the stator 51. This plays a role of preventing the blade 529 and the like from being damaged while allowing the sintered body 541 to abut against an upper surface of the shaft 512, when, with respect to the sintered body 541, for example, an air resistance at the air sending side is increased and a pressure difference between the top and the bottom of the blade 529 is produced, and the blade 529 moves to a side of the rotor 51 by the pressure difference. In addition, bypass openings 529a are formed in the blade 529. When an air resistance at the air sending side rises or an air intake side is blocked, air flows through the bypass openings 529a, and thus, the bypass openings 529a play a role of reducing a pressure difference between the inside and the outside of the blade 529, thereby preventing movements of the blade 529 and the like.

As illustrated in FIG. 10 and FIG. 11, an air receiving port 531 is provided in an upper section of the upper cover 53, and, in a side section thereof, there is formed a half cylinder section 542b which forms the air sending port 542 having a cylindrical shape together with a half cylinder section 542a on a side of the stator 51. Lock openings 533a provided in lock sections 533 which are formed to protrude downward on a side surface of the upper cover 53 and lock projections 543 formed on a side surface of the casing 518 of the stator 51 are engaged with one another, so that the upper cover 53 is fixed to the casing 518 of the stator 51 in a state in which a small space is formed between the upper cover 53 and the blade 529. A stopper 532 which is exposed downwardly is provided in a center of the upper cover 53. When, for example, a state occurs in which the air intake port 531 is blocked or a more upstream side is blocked so that air does not flow into the air intake port 531, the rotor 52 tends to float by a pressure difference between the inside and the outside of the blade 529, and the stopper 532 is for preventing the blade 529 from being damaged by allowing an upper central portion of the blade 529 to abut against the stopper 532 at this moment.

The turbofan 50 includes the above-described configuration, electric power of three-phase pulses is applied to the coil 517, and the rotor 52 rotates according to a cycling frequency of the three-phase pulses.

Here, the turbofan 50 has the configuration in which there is no contact between the stator 51 and the rotor 52 and the air dynamic pressure bearing is arranged between them, and it is a fan which is suitable for high-speed rotation, is small in diameter and lightweight, and may produce a pressure and an air flow amount required as a CPAP device.

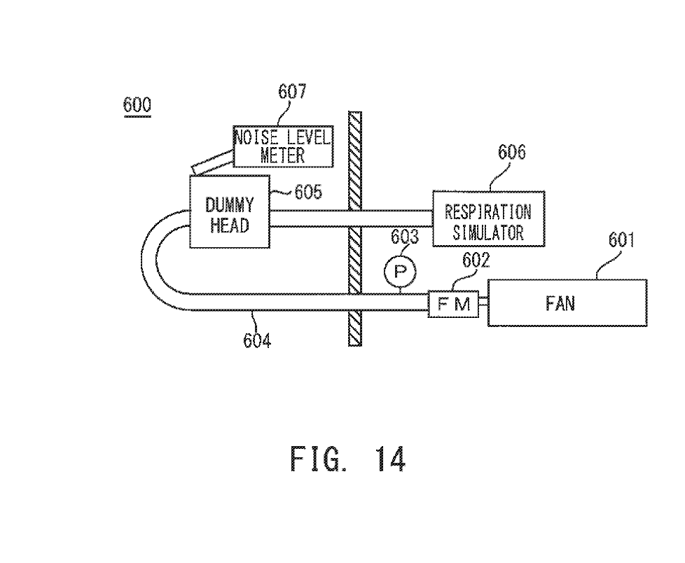

FIG. 14 is a schematic diagram of an experimental equipment.

A dummy head 605 which mimics a shape of a human head and is worn with a mask is placed in an anechoic room 600, and between a fan 601 which is placed outside the anechoic room 600 and the dummy head 605 is coupled by a hose 604 having a length of approximately 2.5 meters. A flowmeter 602 and a manometer 603 are placed at an air output port of the fan 601, and flow amounts and pressures are measured. In addition, a respiration simulator 606 is coupled to the dummy head 605. The respiration simulator 606 has a function to simulate inspiration and expiration and corresponds to a human lung, and a noise level meter 607 is provided near the dummy head 605 (a position corresponding to a human ear), noise when noise simulations are performed by the respiration simulator 606 is measured.

In here, as the fan 601, a fan (blade diameter: approximately 53 mm, weight: approximately 240 g) (Hereafter, the fan will be referred to as “a fan of comparative example” or simply “a comparative example.”) which is incorporated in a commonly commercially available stationary CPAP device, and a fan (blade diameter: 29 mm, weight: approximately 40 g) (Hereafter, the fan will be referred to as “a fan of embodiment example” or simply “an embodiment example.”) which is equivalent to the turbofan used in the present embodiment are used. The fan of embodiment example is basically a fan of the air dynamic pressure bearing configuration, which is explained above with reference to FIGS. 7 to 13.

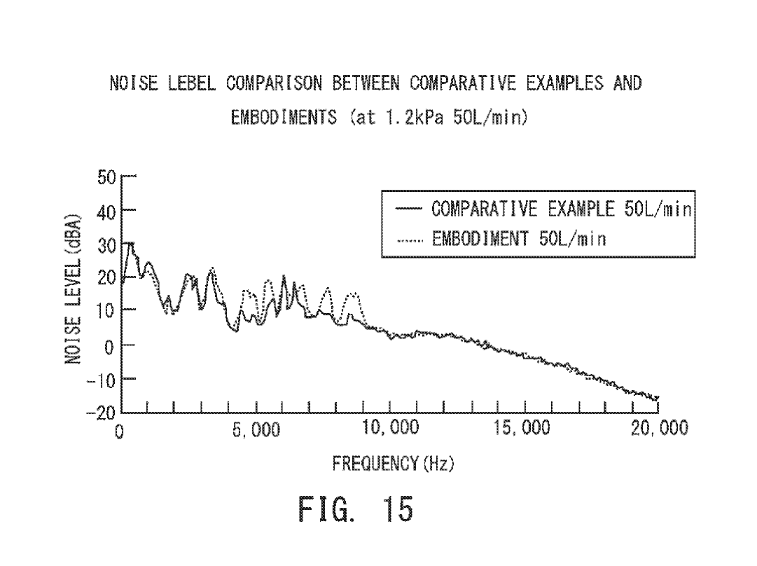

FIG. 15 is a view illustrating noise of the fan of the comparative example and noise of the fan of embodiment example when the pressure is 1.2 kPa and the flow amount is 50 L/min (litter/minute). However, “the fan of embodiment example” is only a fan which is not provided with a silencer. The horizontal axis represents the frequency (Hz), and the vertical axis represents the noise level (dBA). The flow amount 50 L/min corresponds to a period of time when breathing stops (a period of time between an expiration and an inspiration). When sounds in order of 5 kHz to 7 kHz are large, the sounds are tend to be easily sensed as being harsh to one's ears, and it is required to reduce sounds of such frequency band. Looking into the noise levels at 5 kHz to 7 kHz, at the pressure 1.2 kPa and the flow amount 50 L/min (breathing stops) illustrated in the FIG. 15, the noise of the embodiment example is slightly larger than that of the comparative example.

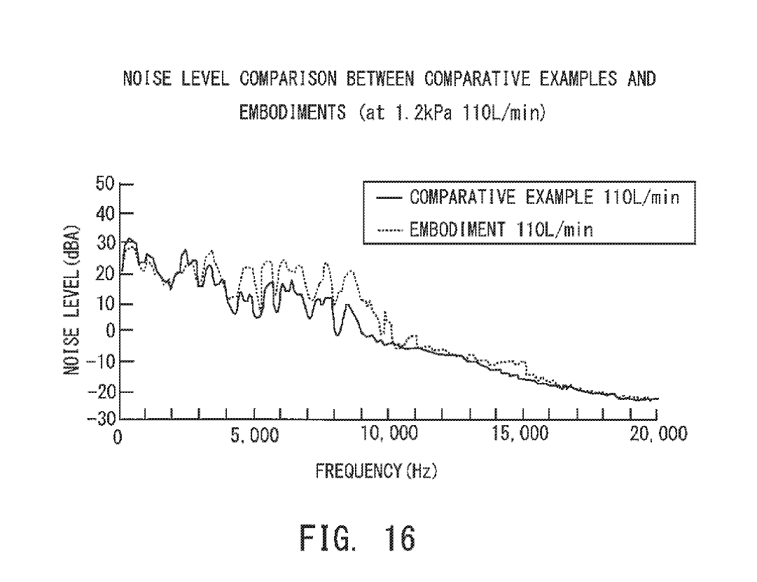

FIG. 16 is a view illustrating noise of the fan of comparative example and noise of the fan of embodiment example when the pressure is 1.2 kPa and the flow amount is 110 L/min. The pressure 1.2 kPa and the flow amount 110 L/min corresponds to a time of inspiration. Also in here, “the fan of embodiment example” is a case in which the fan is only a fan which is not provided with a silencer.

At the pressure 1.2 kPa and the flow amount 110 L/min illustrated in the FIG. 16, the noise of the fan of embodiment example is larger compared to that of the fan of comparative example. In the sense of hearing, a ‘shoo’ sound is heard at the time of inspiration.

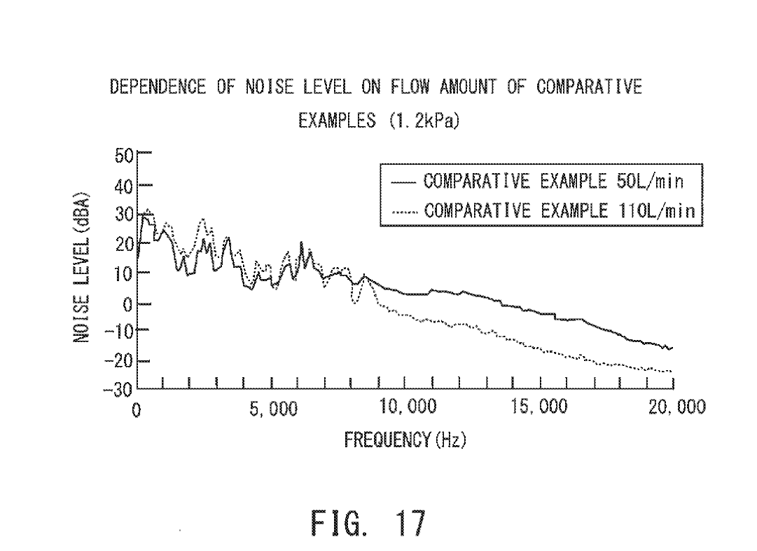

FIG. 17 is a view illustrating noise of the fan of comparative example at the time when breathing stops and at the time of inspiration.

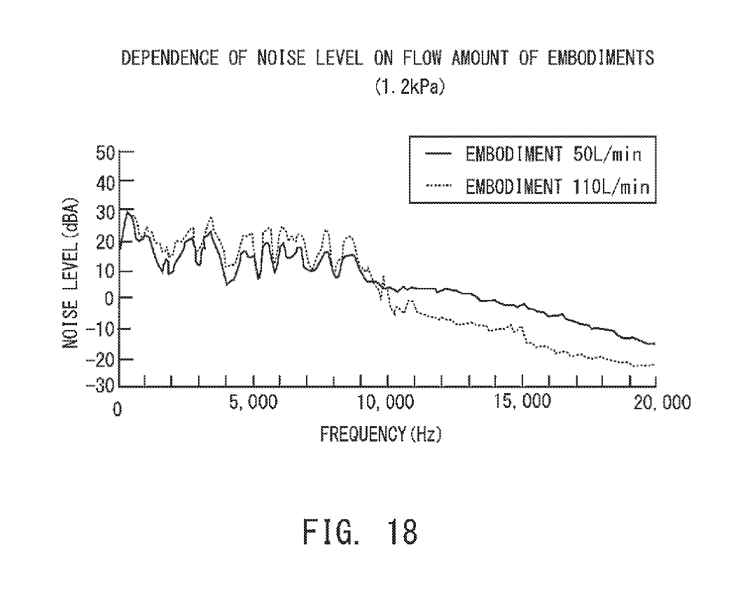

In addition, FIG. 18 is a view illustrating noise of the fan of embodiment example at the time when breathing stops and at the time of inspiration.

Comparing FIG. 17 with FIG. 18, it is found that, with respect to about from 5 kHz to 7 kHz, increasing amounts of the noise at the time of inspiration compared to those at the time when breathing stops are larger in FIG. 18 (the fan of embodiment example) than those in FIG. 17.

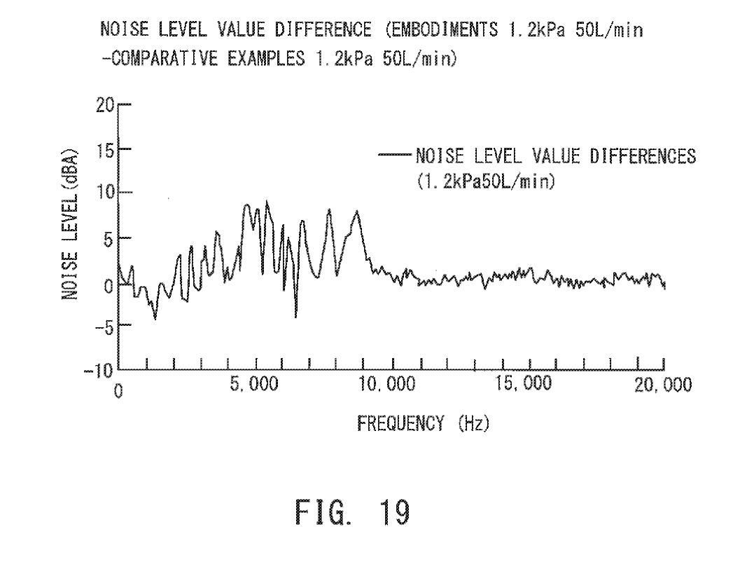

FIG. 19 is a view illustrating differences between the noise levels of the fan of embodiment example and the noise levels of the fan of comparative example at the time when breathing stops. In other words, the FIG. 19 illustrates differences of the two graphs illustrated in FIG. 15.

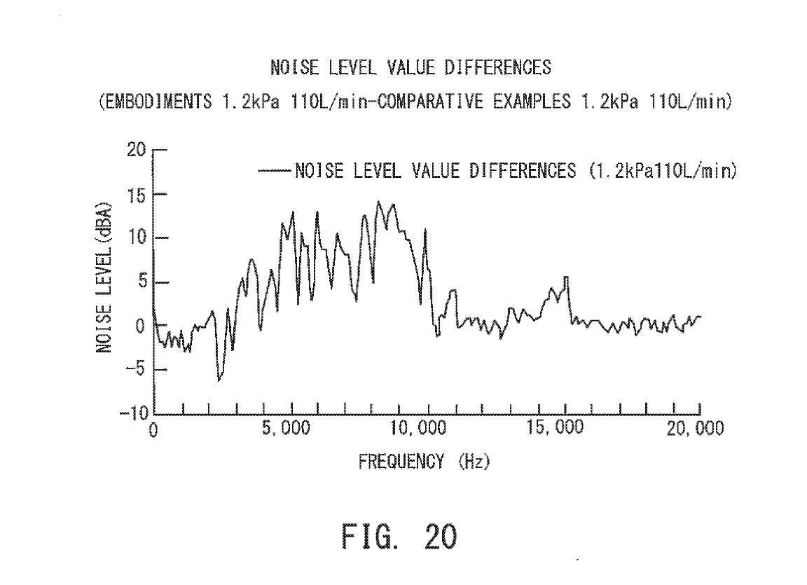

In addition, FIG. 20 is a view illustrating differences between the noise levels of the fan of embodiment example and the noise levels of the fan of comparative example at the time of inspiration. In other words, the FIG. 20 illustrates differences of the two graphs illustrated in FIG. 16.

As seen from these FIG. 19 and FIG. 20, it is found that, at both of at the time when breathing stops (FIG. 19) and at the time of inspiration (FIG. 20), the noise of the fan of embodiment example is larger than that of the fan of comparative example, and specifically larger at the time of inspiration (FIG. 20).

When the fan of embodiment example is applied, compared to a conventional CPAP device in which the fan of comparative example is applied, reductions in size and in weight are achieved significantly, and however, as explained above, it becomes disadvantageous largely in terms of noise. This is because it is required to cause the fan of embodiment example to rotate faster so as to send air of a flow amount same as that of the fan of comparative example according to that the fan of embodiment example is smaller. In addition, it also becomes a disadvantageous factor that changing of the rotation speed of the fan with respect to changing of the flow amount becomes large.

Then, next, experimental data in a case in which an emission silencer is attached at a side of the air sending port of the fan of embodiment example will be introduced.

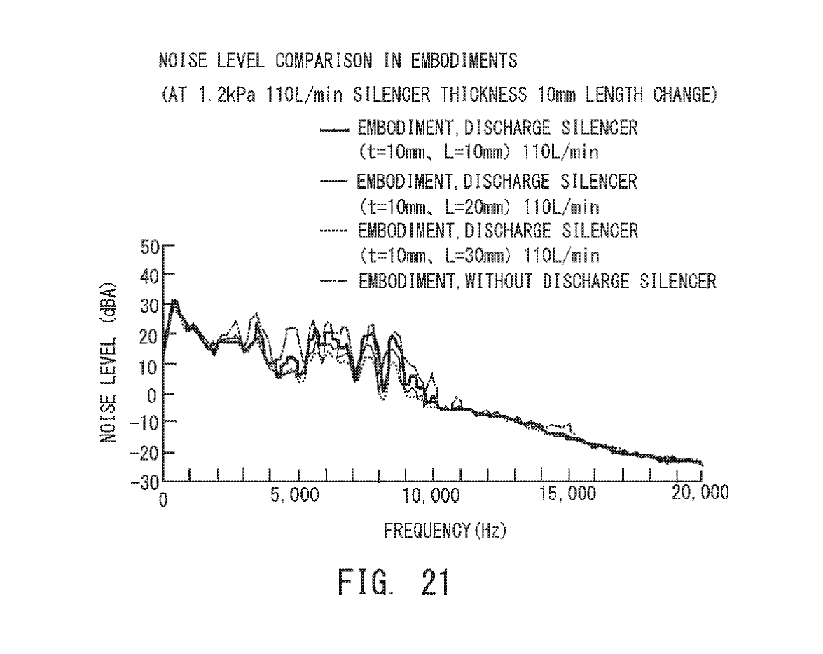

FIG. 21 is a view illustrating changing of the noise levels at the time of inspiration when a length of a sound absorbing material of the emission silencer is changed.

Urethane foam is used for the sound absorbing material in here. The thickness t illustrated in FIG. 5 is t=10 mm, and in the FIG. 21, noise levels when the length L is made to three types of L=10 mm, 20 mm and 30 mm are illustrated. In addition, in the FIG. 21, noise levels when an emission silencer is not applied (see FIG. 16) are also illustrated. The diameter D of the emission flow path is D=12 mm.

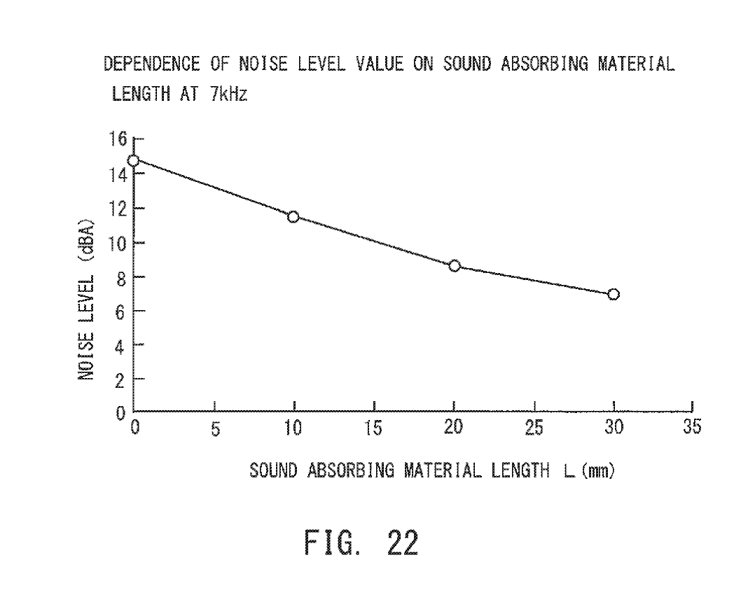

FIG. 22 is a view illustrating noise levels at 7 kHz with respect to the length of the sound absorbing material included in the emission silencer which noise levels are read and obtained from the FIG. 21.

As seen from FIG. 21 and FIG. 22, the longer the length of the sound absorbing material is, the larger the effects of absorbing the sounds become, and thus the noise levels are decreased. More specifically, under the experimental condition illustrated in FIG. 14, when an emission silencer having a length of the order of L=20 mm is provided, it possible to reduce noise more compared to the fan of comparative example.

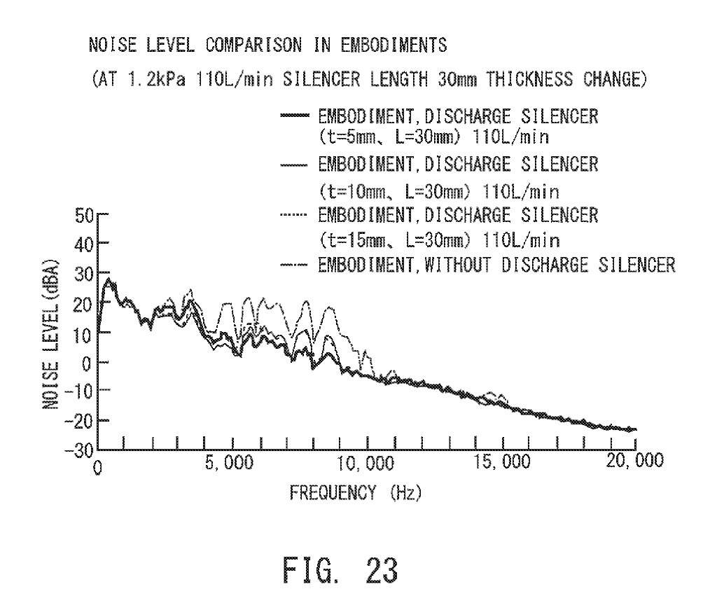

FIG. 23 is a view illustrating changing of the noise levels at the time of inspiration when the thickness of the sound absorbing material of the emission silencer is changed.

For the sound absorbing material, urethane foam is applied as same as the case of FIG. 21. In here, the length L of the sound absorbing material is fixed to L=30 mm, and the thickness t is changed among t=5 mm, 10 mm and 15 mm. In addition, the noise levels (see FIG. 6) when an emission silencer is not provided are also illustrated in here.

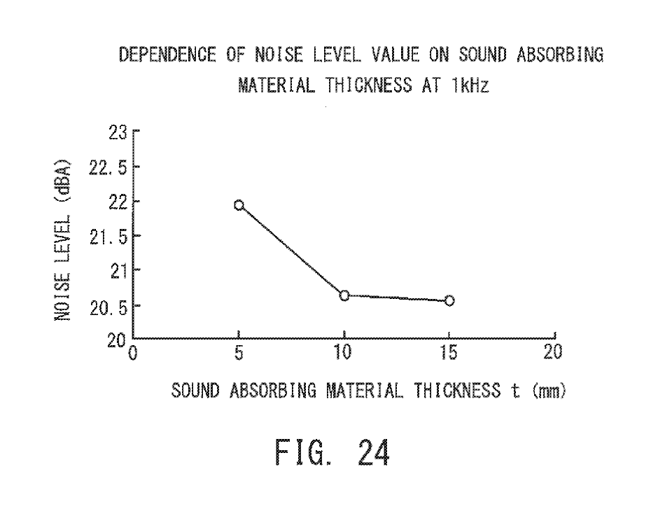

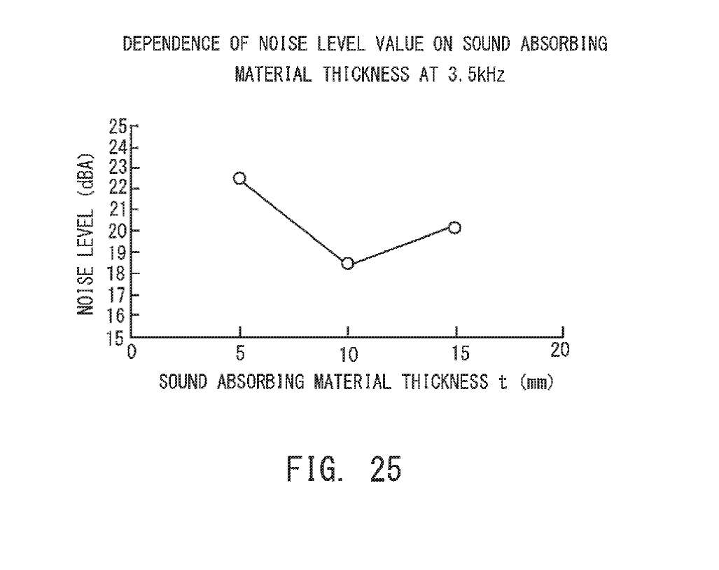

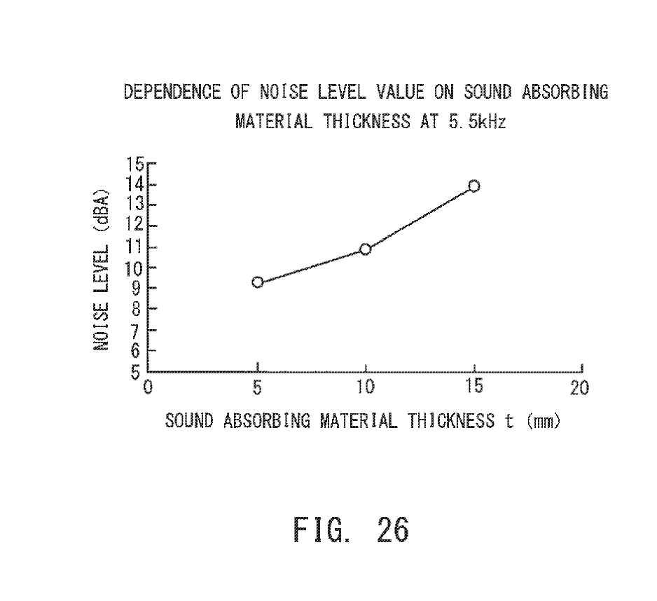

FIGS. 24 to 26 are views illustrating noise levels of 1 kHz, 3.5 kHz and 5.5 kHz which noise levels are read from FIG. 23, respectively.

As seen from these figures, the thinner the thickness of the sound absorbing material is, the larger the effects in which the noise levels of the higher frequencies are reduced become.

Accordingly, when an emission silencer in which a sound absorbing material made of foaming material is applied is used, by adjusting the thickness or the length thereof, it is possible to effectively reduce noise of a targeted frequency band.

In other words, by applying a fan of air dynamic pressure bearing, it is possible to achieve significant reductions in size and in weight, and with respect to noise which become a problem when such a fan of air dynamic pressure bearing is applied, it is possible to effectively reduce the noise by applying an emission silencer. In other words, by a combination of a fan of air dynamic pressure bearing and an emission silencer, it is possible to achieve the compatibility between reductions in size and in weight and noise reduction in a high order.

This ends the explanations of the CPAP device 1A according to the first embodiment, and in the following, embodiments of a second embodiment and embodiments thereafter will be explained. Incidentally, in the drawings illustrating each of the second embodiment and the embodiments after the second embodiment, for the convenience of easy understanding, components and the like functionally corresponding to those included in the CPAP device according to the first embodiment even though there are differences in shapes and the like are illustrated while being assigned with signs same as those put in each of the drawings used for the explanations of the first embodiment, and configuration portions distinctive to each embodiment will be explained.

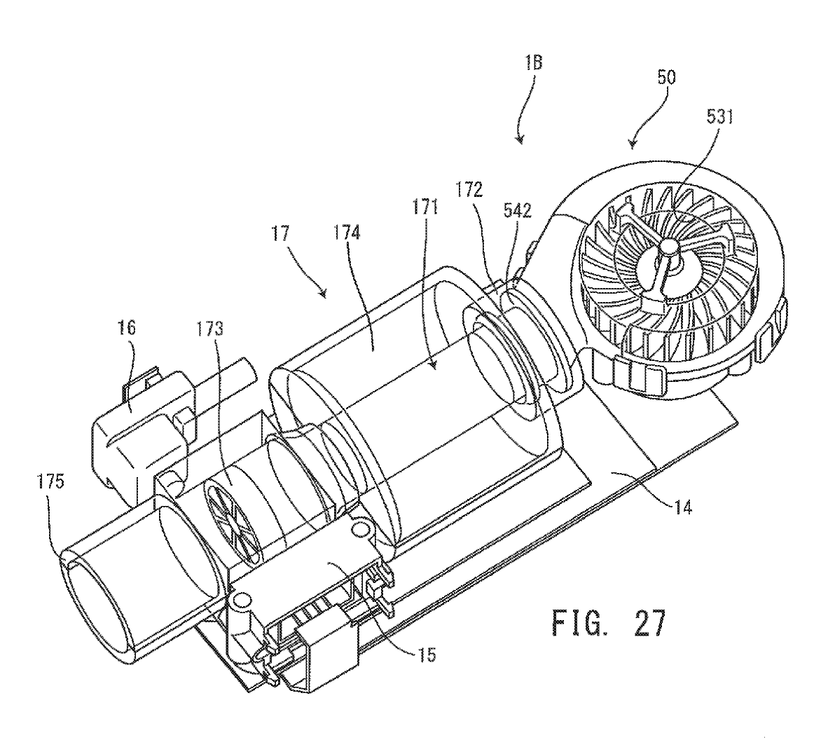

FIG. 27 is a transparent view when a casing and a suction silencer are removed from the CPAP device according to the second embodiment, and a fan, an emission silencer and the like are viewed from obliquely above. This FIG. 27 is a figure corresponding to FIG. 6 which is used for the explanations of the CPAP device according to the first embodiment.

In an emission silencer 17 included in the CPAP device 1B according to the second embodiment, a sound absorbing material 174 is provided on a side of a turbofan 50, and a rectifying element 173 is arranged on a more downstream side in an air flow than the sound absorbing material 174. A flow sensor 15 and a pressure sensor 16 are coupled to a downstream side of the rectifying element 173.

As illustrated in here, either the sound absorbing material 174 or the rectifying element 173 may arranged in the upstream side or the downstream side.

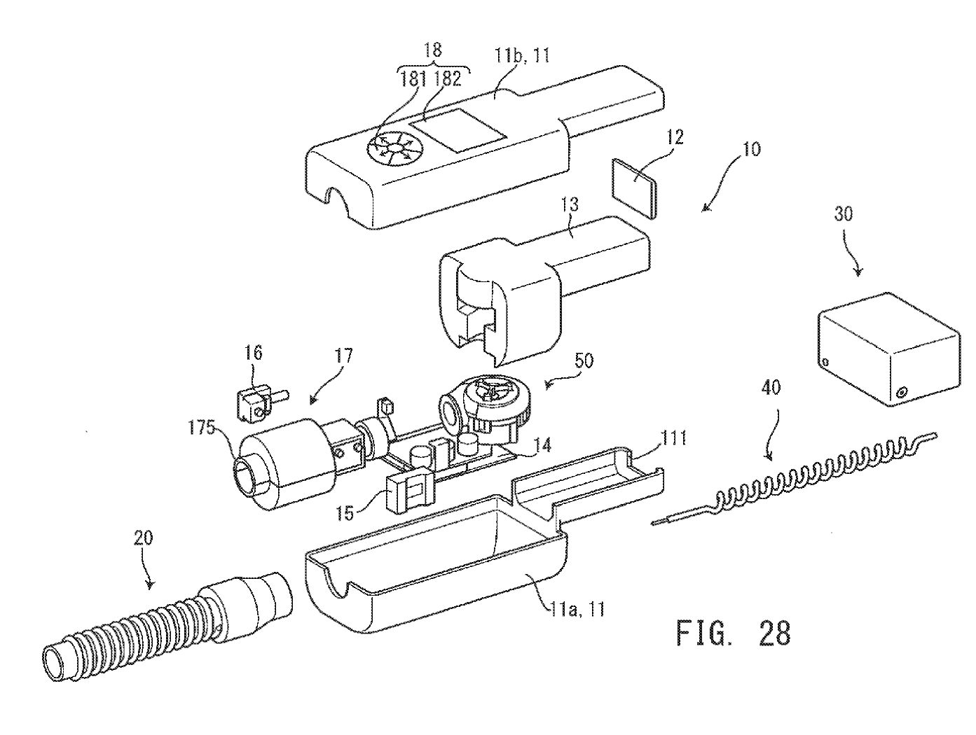

FIG. 28 is an exploded perspective view of a CPAP device according to a third embodiment.

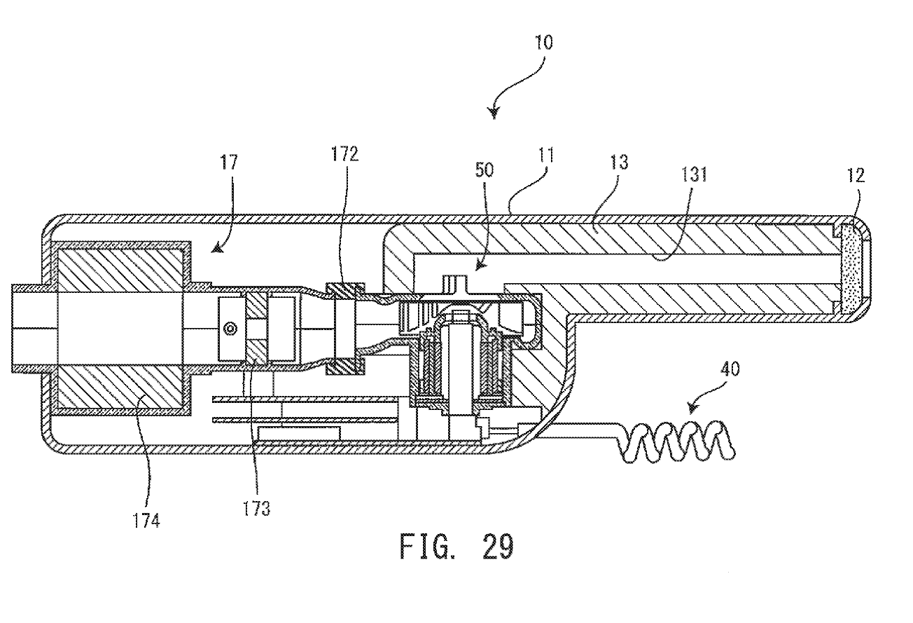

In addition, FIG. 29 is a sectional view of a blower unit of the CPAP device whose exploded perspective view is illustrated in FIG. 28.

A blower unit of the CPAP device 1C according to the third embodiment illustrated in FIG. 28 and FIG. 29 includes a casing 11 which is formed to have a squarish shape compared to the blower unit (see FIGS. 1 and 2) of the above-described CPAP device 1A according to the first embodiment. In the case of the blower unit according to the first embodiment, the blower unit has the casing having the round shape so as to roll according to posture changing of a patient while being in bed, and however, it is assumed in here that the blower unit is placed, for example, on a kakebuton (a bed cover) and the like of a patient while being in bed, and posture stability of the blower unit 10 is considered to be important. When a patient changes its posture by tossing about, turning over and the like, the blower unit 10 according to the third embodiment follows the posture changing of the patient mainly by moving slidably.

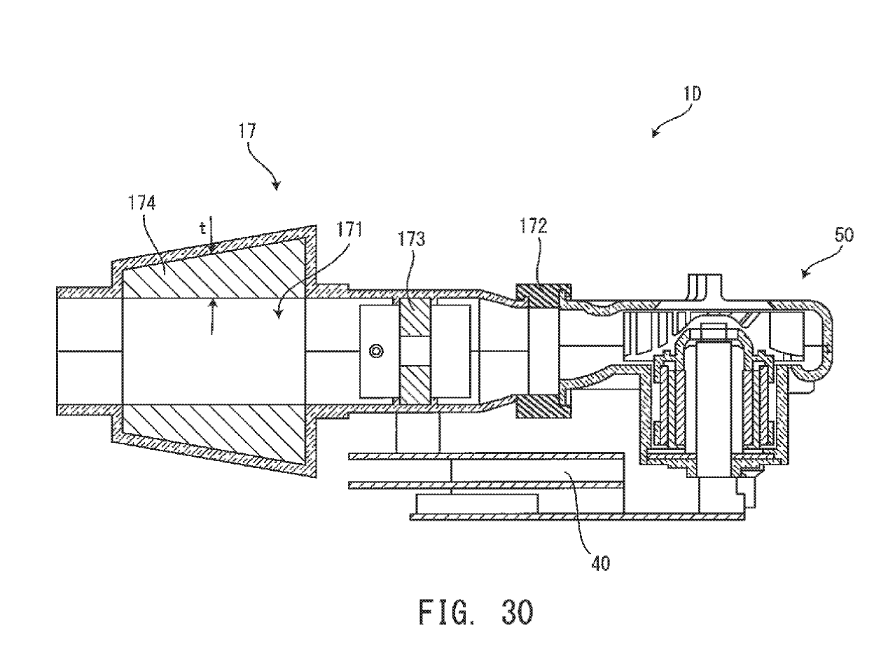

FIG. 30 is a sectional view of a fan and an emission mechanism of a CPAP device according to a fourth embodiment.

In a case of the CPAP device 1D according to the fourth embodiment, a sound absorbing material 174 included in an emission silencer 17 in a blower unit 10 is formed such that the thickness thereof becomes continuously thinner from an upstream side toward a downstream side in an air flow. As easily conjectured from the above-described experimental data, specifically, the experimental data when the thickness t of the sound absorbing material which data is illustrated in FIGS. 23 to 26 is changed, it is expected that noise in a broad frequency band is reduced by changing the thickness t.

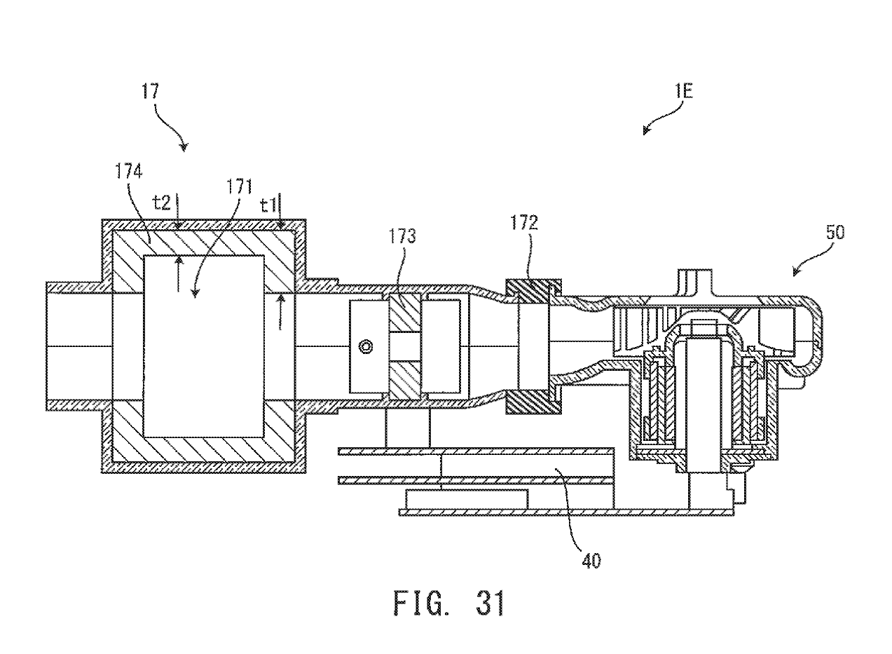

FIG. 31 is a sectional view of a fan and an emission mechanism of a CPAP device according to a fifth embodiment.

In a case of the CPAP device 1E according to the fifth embodiment, a sound absorbing material 174 of an emission silencer 17 in a blower unit 10 has the thickness t which is thick at both end sections (t=t1) and thin at a center section (t=t2). From this, similarly to the case of the fourth embodiment illustrated in FIG. 30, it is expected that noise in a broad frequency band is reduced. In addition, in the case of the emission silencer 17 according to the fifth embodiment, the sectional area of an emission flow path 171 changes between the both end sections and the center section, and also with this, a noise reduction effect is expected.

Incidentally, the examples including the air dynamic pressure bearing have been explained in here, and however, it is expected that one including an oil dynamic pressure bearing also may achieve similar effects.

### REFERENCE SIGNS LIST

1A-1E CPAP device
10 Blower unit
11 Casing
12 Air filter
13 Suction silencer
14 Control board
15 Flow sensor
16 Pressure sensor
17 Emission silencer
18 User interface
20 Hose
30 Battery case
40 Cable
50 Turbofan
51 Stator
52 Rotor
53 Upper cover
111 Air suction port
131 Suction flow path
141 MPU
142 Motor drive circuit
171 Emission flow path
172 Joint
173 Rectifying element
174 Sound absorbing material
175 Air emission port
181 Operation button
182 Display screen
200 Mask
201 Leak opening
300 Patient
301 Battery
302 Connection terminal
511 Shaft base
511a, 514a, 514b, 521a Opening
512 Shaft
512a Upper end section
513 Thrust magnet (inside)
514 Circuit board
515 Connector
516 Coil base
516a Leg section
517 Coil
518 Casing
519 Screw
521 Hub
522 Thrust magnet (outside)
524 Sleeve
525 Magnet
526 Reinforcement ring
527 Back yoke
528 Balance ring
529 Blade
529a Bypass opening
531 Air receiving port
532 Stopper
533 Lock section
533a Lock opening
541 Sintered body
542 Air sending port
542a, 542b Half cylinder section
543 Lock projection
600 Anechoic room
601 Fan
602 Flowmeter
603 Manometer
604 Hose
605 Dummy head
606 Respiration simulator
607 Noise meter

1. A CPAP device comprising:
a blower unit that includes:
a casing which has an air suction port;
a fan which has an air receiving port and an air sending port, is provided with a fluid dynamic bearing, causes air to be suctioned from the air suction port to receive the air from the air receiving port and sends out the air from the air sending port; and
an emission silencer which is coupled to the air sending port and reduces sounds as the air flows which air is sent out from the air sending port by the fan, wherein
an air intake port of a nasal cannula or a mask which has the air intake port, is attached to the head of a patient to cover an external naris or a nose of the patient and supplies air taken in from the air intake port to a respiratory tract of the patient and the blower unit are coupled with a hose, and the air sent out from the blower unit is sent to the nasal cannula or the mask.

2. The CPAP device according to claim 1, wherein the emission silencer is a silencer which includes a sound absorbing material made of foaming material.

3. The CPAP device according to claim 1, further comprising a suction silencer that includes a sound absorbing material in which a suction flow path to guide the air suctioned from the air suction port to the air receiving port is formed, and that supports the fan such that the suction silencer enfolds the fan with the sound absorbing material.

4. The CPAP device according to claim 1, wherein the air sending port and the emission silencer are connected with a joint formed with an elastic body.

5. A blower unit comprising:
a casing which has an air suction port;
a fan which has an air receiving port and an air sending port, is provided with a fluid dynamic bearing, causes air to be suctioned from the air suction port to receive the air from the air receiving port and sends out the air from the air sending port; and
an emission silencer which is coupled to the air sending port and reduces sounds as the air flows which air is sent out from the air sending port by the fan, wherein
the blower unit sends air into a hose which is coupled to an air intake port of a nasal cannula or a mask which has the air intake port, is attached to the head of a patient to cover an external naris or a nose of the patient and supplies the air taken in from the air intake port to a respiratory tract of the patient.
